(19) United States
(12) Patent Application Publication
(10) Pub. No.: US 2021/0215657 A1
(43) Pub. Date: Jul. 15, 2021

(54) SYSTEMS AND METHODS FOR LIQUID AND GAS CLASSIFICATION

(71) Applicant: THE BOARD OF REGENTS OF THE NEVADA SYSTEM OF HIGHER EDUCATION ON BEHALF OF THE UNIVERSITY OF NEVADA,, Las Vegas, NV (US)

(72) Inventors: Robert Rippee, Las Vegas, NV (US); Gina Marano, Las Vegas, NV (US)

(21) Appl. No.: 15/734,157

(22) PCT Filed: Jun. 3, 2019
(86) PCT No.: PCT/US19/35187

§ 371 Date: Dec. 1, 2020

Related U.S. Application Data
(60) Provisional application No. 62/679,513, filed on Jun. 1, 2018.

(51) Int. Cl. G01N 33/14; G06N 20/00; G06Q 30/02

(52) U.S. Cl. CPC G01N 33/146; G06Q 30/0201; G06Q 30/0202; G06N 20/00

(57) ABSTRACT

Methods and systems are disclosed for determining characteristics of a liquid, a headspace above the liquid, and/or a gas within a container.

### CROSS REFERENCE TO RELATED APPLICATION

This application claims priority to U.S. Provisional Application No. 62/679,513 filed on Jun. 1, 2018, the contents of which are incorporated by reference herein, in its entirety and for all purposes.

### BACKGROUND

Wine is a complex beverage because each style, winemaker, and vintage all play a significant role in the outcome in the flavor and quality of the wine. In turn, these flavors and quality are correlated to the value and demand for wines. Thus, there are many factors that wine consumers must consider when looking for a wine that they enjoy. Additionally, there are many characteristics to wine, such as alcohol content, body and finish, that may be difficult for someone new to wine drinking to be able to identify and describe. Indeed, the ability to fully recognize and describe these characteristics takes years of training and practice, as well as significant expense, to fully master. Further, wine drinkers may try many different wines before they find one that they enjoy. Thus, it is difficult for new wine drinkers to be able to fully understand all the different choices of wines available, the structural components contributing to each wine, and how those factors define the flavor characteristics of each wine. These characteristics may then be used to determine other types of wines, producers, and vintages they would prefer.

Additionally, vintage wines are often sold at premium prices to collectors. These wines fetch premium prices due to their rarity and unique characteristics. The value of these wines is directly correlated to their rare and unique characteristics. In the case of these rare wines, fraud can occur when unscrupulous characters misrepresent or present counterfeit wines as these rare vintages. It is essential to the market that the authenticity of these rare wines be established. Currently, opening and sampling these wines by experts is one of the methods used to establish authenticity. Opening a bottle of a rare, vintage decreases its value tremendously. Thus, a means to determine and validate the authenticity of a rare vintage wine based on a miniscule sample withdrawn with a needle or other device would provide great value in the marketplace for rare, vintage wine.

Lastly, the presence of undesirable organic or inorganic compounds can have detrimental effects to both the taste and value of wines, examples include the presence of pesticides, the presence of organic chemicals due to cork rot and other organic processes, the presence of other undesirable chemicals, that taint or adversely affect the taste and value of the wine. These and other shortcomings are addressed by the disclosure herein.

### SUMMARY

It is to be understood that both the following general description and the following detailed description are exemplary and explanatory only and are not restrictive, as claimed. Provided are methods and systems for determining the characteristics of a liquid or headspace above the liquid in a container.

In one embodiment, a system comprises a container containing a sample liquid for classification within the vessel. The system also comprises one or more sensors, wherein the one or more sensors are configured to take a plurality of measurements of the liquid within the container. The system further comprises a controller, in communication with the one or more sensors. The controller is configured to receive, from the one or more sensors, data indicative of the measurements of the liquid. The controller is also configured to determine, based on the received data, a plurality of characteristics of the liquid. The controller is further configured to determine, based on the characteristics of the liquid, an identity of the liquid, wherein the identity comprises the chemical composition of the liquid and a type of the liquid. Additionally, the controller is configured to transmit, to a computing device, the identity of the liquid.

In another embodiment, an apparatus, comprises one or more processors, and a memory storing processor executable instructions that, when executed by the one or more processors, cause the apparatus to receive, from one or more sensors, data indicative of a plurality of measurements of a liquid within a container. The processor executable instructions further cause the apparatus to determine, based on the received data, a plurality of characteristics of the liquid. The processor executable instructions also cause the apparatus to determine, based on the characteristics of the liquid, a chemical signature, standard or profile of the liquid. These characteristics in turn are used via a matching algorithm to determine an identity of the liquid from a global database of known liquids, wherein the identity comprises a type or variety of the liquid and other unique characteristics. Additionally, the processor executable instructions cause the apparatus to transmit, to a computing device, the identity of the liquid.

In a further embodiment, a method comprises receiving, from one or more sensors, data indicative of a plurality of measurements of a liquid within a container. The method also comprises determining, based on the received data, a plurality of characteristics of the liquid. The method further comprises determining, based on the characteristics of the liquid, an identity of the liquid, wherein the identity comprises a type of the liquid, a manufacturer of the liquid, and a year the liquid was manufactured. Additionally, the method comprises transmitting, to a computing device, the identity of the liquid.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a diagram illustrating an exemplary system;

FIG. 2 is a block diagram illustrating an exemplary system;

FIG. 3 is a diagram illustrating an exemplary system;

FIG. 4 is a flowchart illustrating an exemplary method;

FIG. 5 is a flowchart illustrating an exemplary method;

FIG. 6 is a flowchart illustrating an exemplary method;

FIG. 7 is a flowchart illustrating an exemplary method;

FIG. 8 is a flowchart illustrating an exemplary method;

FIG. 9 is a diagram illustrating wine characteristics;

FIG. 10 is a diagram illustrating wine acidity;

FIG. 11 is a diagram illustrating wine sweetness;

FIG. 12 is a diagram illustrating wine alcohol;

FIG. 13 is a diagram illustrating wine body characteristics; and

FIG. 14 is a block diagram illustrating an exemplary computing system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

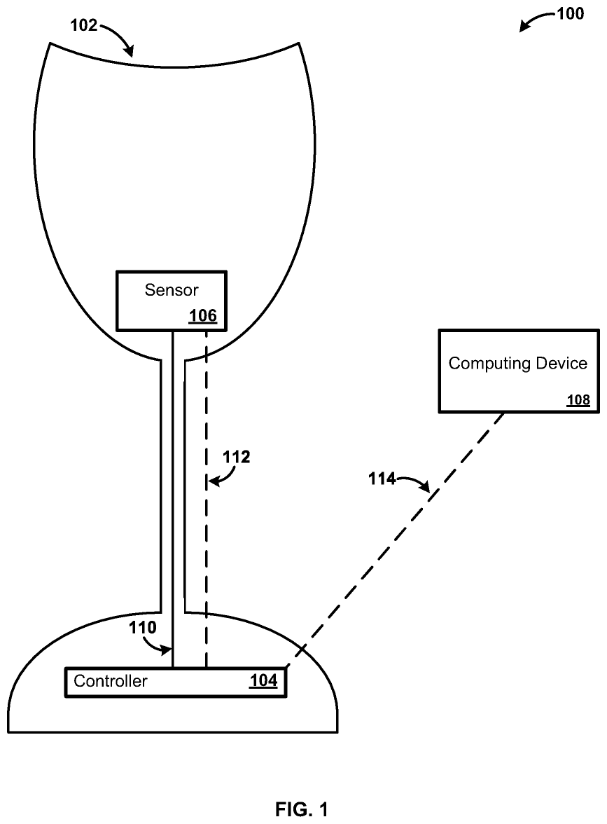

FIG. 1 illustrates a system 100 for remotely and/or automatically measuring characteristics of a liquid. The liquid can include the liquid, the headspace above the liquid, a gas associated with the liquid, and so forth. The system 100 can comprise one or more of a container 102 for holding the liquid, a controller 104, a sensor 106 coupled with the container 102 or independent from the container 102, and a computing device 108. In one exemplary embodiment, the controller 104 comprises a microcontroller. The sensor 106 can be in communication with the controller 104 via a wired communications link 110 and/or a wireless communications link 112. The sensor 106 can be configured to take one or more measurements of the liquid. For example, the sensor 106 can be configured to determine one or more of a sweetness (e.g., sugar content) of the liquid, an acidity of the liquid, a tannin concentration of the liquid, an alcohol content of the liquid, trace organic chemicals within the liquid, the presence of pesticides or other undesirable organic or inorganic chemicals, and/or a body (e.g., viscosity) of the liquid. Further, the characteristics can also relate to the headspace above the liquid and/or a gas associated with the liquid.

The container 102 can be any type of suitable container for holding a liquid. For example, the container 102 can be a wine container (e.g., glass, bottle, etc.), a rocks glass (e.g., container), a beaker, a flask, or any container. The container 102 can be made of any type of material such as glass, plastic, metal, etc. Further, the container 102 can comprise any shape and be any shape.

As shown, the controller 104 is communicatively coupled with the sensor 106 via a wired communications link 110 and/or a wireless communications link 112. The controller 104 can use the communications links 110 and 112 to provide control signals to the sensor 106, as well as received communications from the sensor 106. For example, the communications link 110 can directly couple the controller 104 and the sensor 106 via one or more cables or wires (e.g., communications wires, Universal Serial Bus (USB), Ethernet, etc.). As another example, the communications link 112 is a wireless connection such that the controller 104 communicates wirelessly with the sensor 106. For example, the controller 104 communicates with the sensor 106 via Bluetooth™, Wi-Fi, or any wireless communication standard. The controller 104 can also use the communications links 110 and 112 to provide power to the sensor 106. For example, the controller 104 can have a power supply that is capable of providing power to the sensor 106.

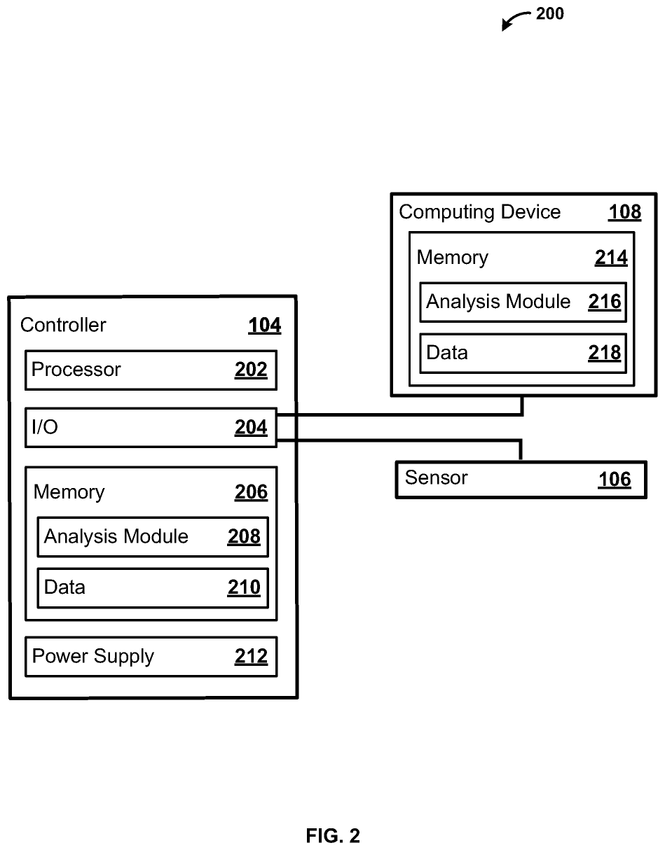

The controller 104 can include a processor, a memory, and an interface for communicating with other devices using wired connections or wirelessly using, for example, Wi-Fi, Bluetooth, cellular service as will be explained in more detail with regards to FIG. 2. In one example, the controller 104 controls the sensor 106. The controller 104 can control the sensor 106 based on data provided by the sensor 106. For example, the controller 104 can receive data from the sensor 106, and the controller 104 can use the data to determine how to control the sensor 106. As another example, the controller 104 can receive data from the sensor 106 and communicate the data to the computing device 108. The controller 104 can also perform an analysis on the data received from the sensor 106. For example, the controller 104 can receive data from the sensor 106, and the controller can make a determination regarding the liquid contained within the container 102, as well as the characteristics of the liquid contained within the container 102. Further, the controller 104 can receive data from the sensor 106, and the controller can make a determination regarding a headspace above the liquid and/or a gas contained within the container 102, as well as the characteristics of the headspace above the liquid and/or gas contained within the container 102. While a single controller 104 is illustrated for ease of explanation, a person skilled in the art would appreciate that any number of controllers may be present in the system 100. Further, while the controller 104 and the sensor 106 are illustrated as separate devices for ease of explanation, a person skilled in the art would appreciate that the controller 104 can include the functionality of the sensor 106 and vice versa.

The sensor 106 can be any suitable sensor for measuring characteristics of a liquid within the container 102, a headspace above the liquid within the container 102, and/or a gas within the container 102. For example, the sensor 106 can be capable of measuring any of a number or organic and inorganic chemicals, a pH of the liquid, an amount of tannins in the liquid, an alcohol content of the liquid (e.g., an average alcohol content), a color of the liquid, a body or viscosity of the liquid, a sweetness of the liquid, a finish of the liquid, a clarity of the liquid, and the aromatic compounds of the liquid. Further, the sensor 106 can be capable of measuring any of the aforementioned measurements of a headspace above the liquid and/or a gas within the container 102. In an example, the sensor 106 is a miniaturized sensor. The sensor 106 can include any sensors or sources for measuring characteristics of the liquid, the headspace, or the gas within the container 102. While a single sensor 106 is shown for ease of explanation, a person skilled in the art would appreciate that the sensor 106 can be more than one sensor. Further, a person skilled in the art would appreciate that the sensor 106 can be capable of taking more than one measurement of the liquid, the headspace, and/or the gas.

The computing device 108 can be any type of electronic device. For example, the computing device 108 can be a computer, a smartphone, a laptop, a tablet, a wireless access point, a server, or any other electronic device. The computing device 108 can include an interface for communicating wirelessly using, for example, Wi-Fi, Bluetooth, cellular service, etc. As illustrated in FIG. 1, the computing device 108 and the controller 104 can be communicatively coupled via a communications link 114. As an example, the computing device 108 and the controller 104 can communicate via a wireless network (e.g., Wi-Fi, Bluetooth™). The computing device 108 and the controller 104 can exchange data using the communications link 114.

The controller 104 can provide data from the sensor 106 to the computing device 108. For example, the controller 104 can transmit the data received from the sensor 106 to the computing device 108. The computing device 108 can use the data transmitted by the controller 104 to determine characteristics of a liquid, the headspace, and/or the gas, within the container 102. As an example, the container 102 may contain a wine. The sensor 106 can determine one or more chemical measurements of the wine (e.g., amounts of organic or inorganic chemicals, pH, alcohol content, sugar content, etc.) that produce data associated with the wine. The sensor 106 transmits the data to the controller 104, which in turn transmits the data to the computing device 108. The computing device 108 can then determine several characteristics (e.g., body, type of wine, sweetness, etc.) of the wine based on the data received from the controller 104. The computing device 108 may also identify the wine based on the characteristics. For example, the computing device 108 can receive the data from the sensor 106, and can compare the data to database that comprises characteristics of known wines. The computing device 108 can then determine a possible identity of the wine, such as type, region, manufacturer, etc., based on a comparison to the database. As another example, the computing device 108 can utilize machine learning to identify the wine based on the data. While wine was used for ease of explanation, a person skilled in the art would appreciate the computing device 108 can be capable of identifying other liquids, the headspaces, and/or the gases within the container 102. In this manner, the computing device 108 can receive the measurements from the sensor 106 and determine the liquid, the headspace, and/or the gas within the container 102. While the above example describes the controller 104 as receiving the data from the sensor 106 and the controller 104 transmitting the data to the computing device 108, a person skilled in the art would appreciate that the sensor 106 can directly communicate with the computing device 108 without needing communicate via the controller 104.

The controller 104 can also determine the current operational status of the sensor 106. For example, the controller 104 can provide data indicating that the 106 is not functioning properly. As another example, the controller 104 can determine data relating to the last time a measurement was performed using the sensor 106. The controller 104 can provide the operational status and/or the last time a measurement was performed to the computing device 108. The computing device 108 can use the provided data to determine an operating status of the sensor 106. While the computing device 108 and the controller 104 are illustrated as directly communicating via the communications link 114, a person skilled in the art would appreciate that the computing device 108 and the controller 104 can communicate via additional devices. For example, the computing device 108 can communicate with a device such as a server, wireless router, and/or access point which in turn communicates with the controller 104.

The computing device 108 can also transmit settings or instructions to the controller 104 to manage operation of the controller 104. For example, the computing device 108 can provide software to the controller 104 that provides instructions for data collection using the sensor 106. The computing device 108 can also transmit settings to the controller 104 that indicate how the controller 104 should operate. For example, the computing device 108 can provide the controller 104 with power management settings for the controller 104 and/or the sensor 106. The computing device 108 can also transmit settings to the controller 104 that indicate when the controller 104 should provide data to the computing device 108. For example, the computing device 108 can indicate start and stop times that the controller 104 should taking measurements using the sensor 106. As another example, the computing device 108 can indicate times that the controller 104 should start dynamically controlling the sensor 106. For example, the controller 104 can start dynamically controlling the sensor 106 when a liquid, a headspace above a liquid, and/or a gas is added to the container 102. A user of the computing device 108 can actively select an instruction or settings that are transmitted to the controller 104. For example, the user can utilize a user interface associated with the computing device 108, and the user can select one or more commands via the user interface to control operation of the controller 104. After receiving the input from the user, the computing device 108 can provide the instructions and/or settings to the controller 104. For example, the computing device 108 can automatically transmit, via the communications link 114, instructions to the controller 104 based on the user indicated preferences and/or settings that were received via the user interface. The computing device 108 can also dynamically decide the instructions or settings that are transmitted to the controller 104 without input from a user. In another example, the computing device 108 receives input from a user indicating the preferences and/or settings the user would like the computing device 108 to implement.

The computing device 108 can also transmit settings or instructions to the controller 104 to manage how the controller 104 controls the sensor 106. For example, the computing device 108 can transmit settings to the controller 104 that indicate the timing of how the controller 104 should take measurements using the sensor 106 in order to measure characteristics of the liquid, the headspace, and/or the gas within the container 102. For example, the computing device 108 can indicate start and stop times that the controller 104 should activate the sensor 106. The computing device 108 can also indicate times that the controller 104 should start dynamically controlling the sensor 106. For example, the controller 104 can dynamically control the sensor 106 when a liquid, a headspace above a liquid, and/or a gas is added to the container 102. As a further example, the computing device 108 can indicate how the controller 104 should provide data to the computing device 108 from the sensor 106. In one example, a user of the computing device 108 actively selects the instructions or settings that are transmitted to the controller 104. In another example, the computing device 108 dynamically decides the instructions or settings that are transmitted to the controller 104 without input from a user. In another example, the computing device 108 receives input from a user indicating the preferences and/or settings the user would like the computing device 108 to implement. The computing device 108 can then automatically transmit instructions to the controller 104 based on the user indicated preferences and/or settings. In one example, the user of the computing device 108 selects specific settings for the sensor 106.

As a further example, the computing device 108 can provide a control signal to the controller 104 in order to control operation of the sensor 106. The control signal can include settings for the sensor 106, data related to settings of the sensor 106, instructions for the sensor 106, and any information related to the control of the sensor 106. The computing device 108 can transmit a control signal to the controller 104 to activate the sensor 106. For example, the computing device 108 can send a control signal to the controller 104, via the communications link 114, to initiate a measurement using the sensor 106. The measurement can comprise using the sensor 106 to measure one or more characteristics of the liquid, the headspace, and/or the gas within the container 102.

The computing device 108 can be a personal computing device (e.g., a laptop, a smartphone, a computer, etc.) that has an application which controls the functionality of the controller 104 and/or the sensor 106. For example, the computing device 108 can have data analysis software which controls operation of the controller 104 and the sensor 106 in order to produce the desired data. In another example, the computing device 108 is a smart device that has an application for controlling the controller 104 and/or the sensor 106. In this manner, the computing device 108 is capable of controlling the controller 104 and the sensor 106.

As will be appreciated by one skilled in the art, the communications links shown in FIG. 1 can be, but need not be, concurrent. For example, the communications links for each of the individual communications connections 110, 112, and 114 can be established at a first time and then later terminated. Further, a person skilled in the art would appreciate that any number of computing devices 108, controllers 104, and sensors 106 can be implemented in the system 100.

FIG. 2 shows an exemplary system 200 comprising the controller 104, the sensor 106, and the computing device 108. As shown, the controller 104 comprises a processor 202, an input output interface (I/O) 204, a memory 206, and a power supply 212. In some examples, the controller 104 can include additional parts such as global positioning system (GPS), motion detectors, sensors, and so forth. While a single processor 202 is shown for ease of explanation, a person skilled in the art would appreciate that the controller 104 can include any number of processors 202. Further, the controller 104 can comprise one or more microcontrollers.

The processor 202 can perform various tasks, such as retrieving information stored in the memory 206, and executing various software modules. For example, the processor 202 can execute the analysis module 208 (explained in more detail below) that provides instructions and/or settings to the sensor 106. As an example, the analysis module 208 can provide instructions and/or settings for the sensor 106 measure characteristics of a liquid, the headspace, and/or the gas. In one example, the processor 202 can be a microcontroller. The processor 202 can be capable of executing any form of firmware and/or software.

As shown, the controller 104 is communicatively coupled via the I/O 204 with the computing device 108 and the sensor 106. The I/O 204 can include any type of suitable hardware for communicating with devices. For example, the I/O 204 can include direct connection interfaces such as Ethernet and Universal Serial Bus (USB), as well as wireless communications, including but not limited to, Wi-Fi, Bluetooth™ cellular, Radio Frequency (RF), and so forth. Further, the I/O 204 can include a multiplexer for amplification, filtering, and/or digitization of signals. For example, the multiplexer can amplify, filter, and digitize the signals provide to the sensor 106 and/or received from the sensor 106.

The sensor 106 can comprise any sensor capable of detecting characteristics of a liquid, the headspace, and/or the gas. For example, the sensor 106 can be configured to determine at least one of the presence of specific organic or inorganic chemicals (e.g., pesticides, cork, chemicals associated with a rotting cork, etc.), a pH of the liquid, an amount of tannins in the liquid, an alcohol content of the liquid, a color of the liquid, a body of the liquid, a sweetness of the liquid, a finish of the liquid, a clarity of the liquid, and/or an aroma of the liquid. Further, the sensor 106 can be configured to determine the aforementioned items for a headspace above the liquid, as well as a gas within the container 102. While specific examples related to wine are provided for ease of explanation, a person skilled in the art would appreciate the sensor 106 can be configured to determine any characteristic of any liquid, headspace, and/or gas. Further, while a single sensor 106 is shown for ease of explanation, a person skilled in the art would appreciate that any number of the sensors 106 could be used. For example, a respective sensor 106 could be configured to determine one of the aforementioned characteristics.

The memory 206 includes an analysis module 208 and data 210. The memory 206 can comprise computer readable media in the form of volatile memory, such as random access memory (RAM), and/or non-volatile memory, such as read only memory (ROM). The memory 206 can also comprise other removable/non-removable, volatile/non-volatile computer storage media. The memory 206 can provide non-volatile storage of computer code, computer readable instructions, data structures, program modules, and other data for the controller 104. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like. The memory 206 can store software that is executable by the processor 202, including operating systems, applications, and/or related software.

The memory 206 can store the data 210. The data 210 can include data received from the sensor 106, settings or preferences for the sensor 106 and/or the controller 104, or any suitable type of data. As an example, the data 210 can include data related to the characteristics of the liquid, the headspace, and/or the gas measured by the sensor 106. As another example, the data 210 can include data derived from the signals output by the sensor 106. While not shown, a person skilled in the art would appreciate that the memory 206 can also include additional software and/or firmware for operating the controller 104.

The controller 104 comprises a power supply 212. The power supply 212 can be any suitable device for providing power to the controller 104. The power supply 212 can also provide power to the controller 104 and the sensor 106. For example, the power supply 212 can include a battery (e.g., Lithium-Ion, alkaline), a direct power connection (e.g., wired) to an external source (e.g., 120 V, 240 V, etc.), and/or a wireless power connection (e.g., induction). The power supply 212 can comprise a voltage regulator configured to provide a constant voltage to the controller 104, as well as to the sensor 106. The power supply 212 can also have a stable current source to provide stable current to the controller 104, as well as to the sensor 106. Thus, the power supply 212 can provide a constant voltage and a stable current to both the controller 104 and the sensor 106.

The power supply 212 can be a battery providing sufficient electrical power (e.g., voltage, current, etc.) for the controller 104 to operate, as well as sufficient power to operate the sensor 106. In this manner, the controller 104 and the sensor 106 can be untethered from other electronic devices in order to allow freedom of movement of the container 102 that the controller 104 and the sensor 106 are coupled with. Further, as will be appreciated by one skilled in the art, the power supply 212 can include additional elements such as a voltage regulator, amplifiers, filters, and so forth. While a single power supply 212 is illustrated for ease of explanation, a person skilled in the art would appreciate additional power supplies 212 may be present that may include similar or different power sources.

The analysis module 208 can include the capability to operate the sensor 106. For example, the analysis module 208 includes the capability to communicate with the sensor 106 and provide operational instructions and/or preferences to the sensor 106. As an example, the analysis module 208 can provide control signals to the sensor 106 to determine measurement of a liquid, a headspace, and/or a gas. For example, the analysis module 208 can provide signals to the sensor 106 to activate and record a specific measurement. As an example, the analysis module 208 can send a signal to the sensor 106 that indicates the sensor 106 should measure an alcohol content of the liquid, the headspace, and/or the gas. The sensor 106 can take a measurement that indicates the alcohol content of the liquid, the headspace, and/or the gas; and the sensor 106 can provide the measurement to the analysis module 108. The measurement can be data, a signal, or any communication capable of indicating the measurement.

The analysis module 208 can provide control signals to the sensor 106 based on the output of the sensor 106. For example, the analysis module 208 can receive output signals and/or data from the sensor 106, and the analysis module 208 can use the output signals and/or data to determine how the sensor 106 should be controlled. In this manner, the analysis module 208, and by direct correlation the controller 104, can control the sensor 106 in a dynamic manner depending on the circumstances surrounding the measurement. For example, the analysis module 208 can instruct the sensor 106 to perform certain measurements based on the liquid, the headspace, and/or the gas; as well as weigh certain measurements differently. As an example, if the analysis module 208 determines from the measurements of the liquid, the headspace, and/or the gas that the liquid is a white wine, the analysis module 208 can instruct the sensor 106 to determine the sweetness of the white wine first before any other measurements because the sweetness of a white wine is rather important comparatively speaking to a red wine. Further, the analysis module 208 can weigh the sweetness of the white wine more importantly than other characteristics because the sweetness of the white wine is very important to drinkers of white wine. As an example, a white wine drinker may prefer chardonnay that is aged in steel barrels which is tarter and has less sweetness than a Riesling even though both are white wines that might be easily confused by a visual inspection of the wine. Thus, the analysis module 208 can dynamically weigh different characteristics of the liquid.

As shown, the computing device 108 comprises memory 214. The memory 214 includes an analysis module 216 and data 218. The memory 214 typically comprises a variety of computer readable media. As an example, the memory 214 can comprise computer readable media in the form of volatile memory, such as random access memory (RAM), and/or non-volatile memory, such as read only memory (ROM). The memory 214 can provide non-volatile storage of computer code, computer readable instructions, data structures, program modules, and other data for the computing device 108. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

The memory 214 can store software that is executable by a processor (not shown), including operating systems, applications, and/or related software. The memory 214 also includes data 218. The data 218 can include data received from the sensor 106, settings or preferences for the controller 104 and/or the sensor 106, or any suitable type of data. As an example, the controller 104 receives data from the sensor 106 via the I/O 204, and then the controller 104 provides the data to the computing device 108 via the I/O 204. The data 218 can include data related to the characteristics of the liquid, the headspace, and/or the gas measured by the sensor 106. As another example, the data 218 can include data derived from the signals output by the sensor 106. For example, the controller 104 can receive one or more signals from the sensor 106 via the I/O 204. The controller 104 can process the received one or more signals to determine data associated with the one or more signals. The controller 104 can then provide the data associated with the one or more signal so the computing device 108 via the I/O 204. While not shown, a person skilled in the art would appreciate that the memory 214 can also include additional software and/or firmware for operating the computing device 108.

The analysis module 216 can include the capability to communicate with the controller 104 and/or the sensor 106, and provide operational instructions and/or preferences to controller 104 and/or the sensor 106. As an example, the analysis module 216 transmits operational instructions and/or preferences to the controller 104 via the I/O 204. The controller 104 in turn transmits the operational instructions and/or preferences to the sensor 106 via the I/O 204. As another example, the analysis module 216 can provide control signals via the controller 104 to the sensor 106 to determine one or more measurements of a liquid, a headspace, and/or a gas. For example, the analysis module 216 can provide signals to the sensor 106 via the controller 104 to activate and record a specific measurement. As an example, the sensor 106 may record the alcohol content of the liquid, the headspace, and/or the gas; or any characteristic of the liquid, the headspace, and/or the gas.

The analysis module 216 can provide control signals to the sensor 106 based on the output of the sensor 106. For example, the analysis module 216 can receive output signals and/or data from the sensor 106 via the controller 104, and the analysis module 216 can use the data to determine how the sensor 106 should be controlled. In this manner, the analysis module 216, and by direct correlation the computing device 108, can control the sensor 106 in a dynamic manner depending on the circumstances surrounding the measurement. For example, the analysis module 216 can instruct the sensor 106 to perform certain measurements based on the liquid, the headspace, and/or the gas, as well as weigh certain measurements differently. As an example, if the analysis module 216 determines from the measurements of the liquid, the headspace, and/or the gas that the liquid is a white wine, the analysis module 216 can instruct the sensor 106 to determine the sweetness of the white wine first before any other measurements because the sweetness of a white wine is rather important comparatively speaking to a red wine. Further, the analysis module 216 can weigh the sweetness of the white wine more importantly than other characteristics because the sweetness of the white wine is very important to drinkers of white wine. As an example, a white wine drinker may prefer chardonnay that is aged in steel barrels which is tarter and has less sweetness than a Riesling even though both are white wines that might be easily confused by a visual inspection of the wine. Thus, the analysis module 216 can dynamically weigh different characteristics of the liquid.

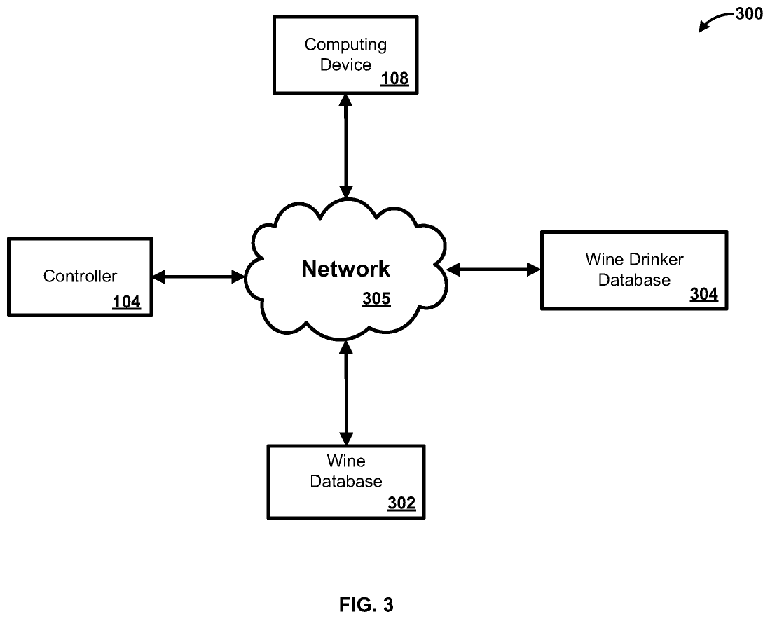

FIG. 3 shows an example of a system 300 in which the present methods and systems may operate. The system 300 comprises one or more controllers 104, one or more computing devices 108, a wine database 302, and a wine drinker database 304. The controller 104, the computing device 108, the wine database 302, and the wine drinker database 304 can be in communication via a private and/or public network 305 such as the Internet, a local area network, and/or a mesh network. Those skilled in the art will appreciate that the present methods may be used in systems that employ both digital and analog equipment. Further, one skilled in the art will appreciate that provided herein is a functional description and that the respective functions may be performed by software, hardware, or a combination of software and hardware.

The wine database 302 can comprise characteristics of a plurality of wines. For example, the characteristics can comprise at least one of: specific organic or inorganic chemicals, a pH of the wine, an amount of tannins in the wine, an alcohol content of the wine, a color of the wine, a body of the wine, a sweetness of the wine, a finish of the wine, a clarity of the wine, and/or aromatic compounds of the wine. The wine database 302 can comprise these characteristics for a plurality of wines. Further, the wine database 302 can include information comprising the type of wine, the producer of the wine, and the vintage. In this manner, the wine database 302 comprises all the necessary information to identify a wine based on the characteristics of the wine. While several characteristics of a wine are described for ease of explanation, a person skilled in the art would appreciate that any characteristics may be stored in the wine database 302.

In one example, the controller 104 and/or the computing device 108 add information to the wine database 302. For example, the wine database 302 may not have information of a specific type of wine that the controller 104 and/or the computing device 108 have measured using the sensor 106. Thus, the controller 104 and/or the computing device 108 can provide the database 302 with the characteristics of the wine. Further, a user of the controller 104 and/or the computing device 108 can provide the information of the type of wine, the manufacturer, and/or the year the wine was produced. For example, the controller 104 and/or the computing device 108 can prompt the user to enter information related to the manufacture of the wine after determining the wine database 302 does not contain information on that specific wine. The user can gather this information off the bottle from which the wine was poured. In this manner, new entries can be added to the wine database 302 as necessary.

In another example, the controller 104 and/or the computing device 108 can measure, using the sensors 106, a small amount of liquid. For example, with rare or vintage wines that are extremely expensive, a miniscule sample of a wine may be withdrawn from the wine bottle with a needle or other device to maintain the seal of the wine bottle by the cork without needing to fully open the wine bottle. The controller 104 and/or the computing device 108 can provide the database 302 with the characteristics of wine, as well as indicate that the wine is a rare or vintage wine. Further, a user of the controller 104 and/or the computing device 108 can provide the information of the type of wine, the manufacturer, and/or the year the wine was produced. The database 302 can compare the characteristics of the wine with the information provided by the user to confirm the authenticity of the wine. That is, the database 302 can determine whether the wine is an authentic rare or vintage wine, and the database 302 can provide a notification to the controller 104 and/or the computing device 108 indicating the authenticity of the wine.

In yet another example, the controller 104 and/or the computing device 108 can measure, using the sensor 106, chemical of a liquid within the container 102. For example, the liquid could be wine, and the sensor 106 can determine whether there are any chemicals that should not be present in the wine, such as pesticides, chemicals due to cork rot, or any other chemicals that can negatively impact the taste and/or value of the wine. Thus, the controller 104 and/or the computing device 108 can determine and/or identify any undesirable chemicals within the liquid.

The wine database 302 can store one or more standards. For example, the wine database 302 can store standards related to a quality of a wine and/or standards related to the identification of the wine. As an example, a standard for champagne can be that grapes have to be grown in the champagne region of France and the percentage mixture of grapes meets a certain threshold. The wine database 302 can utilize the standards to determine whether or not characteristics of a liquid, a headspace, and/or a gas meets those thresholds. Additionally, a device (e.g., the controller 104 and/or the computing device 108) can compare the characteristics of the liquid, the headspace, and/or the gas to the standard to determine whether or not the liquid, the headspace, and/or the gas meets the standard.

The wine drinker database 304 can be a database storing any information associated with a wine drinker. The wine drinker database 304 can store information for a plurality of wine drinkers. For example, the wine drinker database 304 can create and/or store a profile for each of the plurality of wine drinkers. The profile information can include personal information, demographic information, wine preferences, and so forth. For example, the wine drinker database 304 can keep track of all wines that a specific user has consumed or entered into the database. The controller 104 and/or the computing device 108 can provide the wine drinker database 304 with information associated with the wine that is within the container 102 that is associated with the user. As an example, if a user is consuming a pinot noir, as determined from the wine database 302, the controller 104 and/or the computing device 108 can provide this information to the wine drinker database 304 to update the profile of the user. Further, the user may provide the controller 104 and/or the computing device 108 with a rating of the wine so as to determine whether the user liked or disliked the wine.

The wine drinker database 304 can use information associated with a user (e.g., a wine drinker) to suggest a wine that the user may like. For example, the wine drinker database 304 can use machine learning to learn/identify what a user likes and dislikes based on the feedback the user provides for a specific wine that the user has tasted. As an example, if a user indicates that the user strongly dislikes a cabernet sauvignon, the wine drinker database 304 can store this information. Thus, in the future, if the user requests a recommendation for a wine from the wine drinker database 304, the wine drinker database 304 may not provide a recommendation of a cabernet sauvignon since the user previously indicated they do not like that type of wine. As an alternative, the wine drinker database 304 can provide a recommendation for a milder wine such a merlot to see if the user enjoys the merlot. Additionally, the wine drinker database 304 can make a recommendation based on similar types of wine. For example, if a drinker enjoys merlots, the wine database 304 may suggest trying a pinot noir because the wines have similar characteristics even though a merlot and pinot noir are different styles of wine. In this manner, the wine drinker database 304 is capable of learning what types of wine a user enjoys based on the feedback of the user.

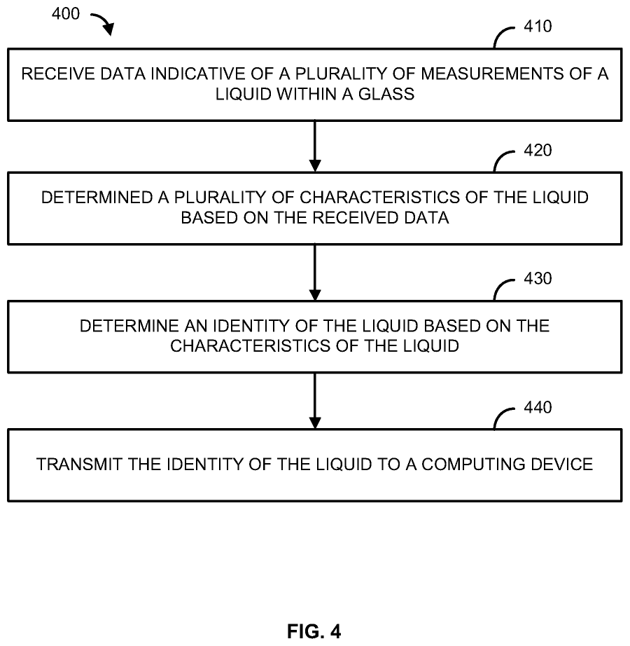

FIG. 4 is a flowchart of an example method 400. At step 410, data indicative of a plurality of measurements of a liquid, a headspace above the liquid, and/or a gas within a container is received. For example, the controller 104 and/or the computing device 108 can receive data from the sensor 106.

At step 420, a plurality of characteristics of the liquid, the headspace, and/or the gas is determined based on the received data. For example, the controller 104 and/or the computing device 108 can determine the characteristics of the liquid, the headspace, and/or the gas. As an example, the controller 104 and/or the computing device 108 can determine at least one of: specific organic or inorganic chemicals in the liquid, a pH of the liquid, an amount of tannins in the liquid, an alcohol content of the liquid, a color of the liquid, a body of the liquid, a sweetness of the liquid, a finish of the liquid, a clarity of the liquid, and the aroma of the liquid. As another example, the controller 104 and/or the computing device 108 can determine at least one of: specific organic or inorganic chemicals in the headspace above the liquid, a pH of the headspace above the liquid, an amount of tannins in the headspace above the liquid, an alcohol content of the headspace above the liquid, a color of the headspace above the liquid, a body of the headspace above the liquid, a sweetness of the headspace above the liquid, a finish of the headspace above the liquid, a clarity of the headspace above the liquid, and the aroma of the headspace above the liquid. As a further example, the controller 104 and/or the computing device 108 can determine at least one of: specific organic or inorganic chemicals in the gas, a pH of the gas, an amount of tannins in the gas, an alcohol content of the gas, a color of the gas, a body of the gas, a sweetness of the gas, a finish of the gas, a clarity of the gas, and the aroma of the gas.

At step 430, an identity of the liquid, the headspace, and/or the gas is determined based on the characteristics of the liquid, the headspace, and/or the gas. The identity of the liquid can be the chemical composition of the liquid, the type of the liquid, a chemical signature of the liquid, a profile of the liquid, a standard of the liquid, and/or any identifying characteristic of the liquid. For example, the controller 104 and/or the computing device 108 can provide the characteristics to the wine database 302. The wine database 302 can to determine the identity of the wine based on the characteristics of the liquid, the headspace, and/or the gas. For example, the wine database 302 can receive the characteristics and can search the data entries of the wine database 302 to determine if there are any matches for the characteristics. That is, the wine database 302 can determine any wines that have similar characteristics as the measured characteristics. Similar characteristics can include identical characteristics, some characteristics being identical and some being similar, comparable characteristics, and so forth. If there is a match, the wine database 302 provides the identity of the matched wine to the controller 104 and/or the computing device 108. If there is not a match, the wine database 302 may indicate to the controller 104 and/or the computing device 108 that the liquid has not be previously identified. In turn, the controller 104 and/or the computing device 108 can prompt the user to identify the liquid so an entry can be added to the wine database 302. While the term match has been used for ease of explanation, a person skilled in the art would appreciate that a match does not necessarily mean an exact match. For example, if the characteristics are within a statistically acceptable likelihood of a wine previously identified within the wine database 302, the unidentified wine can still be considered a match with the identified wine. Stated differently, the wine database 302 is capable of matching liquids with previously identified liquids even if they are not 100% matches as there are typically statistically significant variation from one bottle of liquid to another.

At step 404, the identity of the liquid, the headspace, and/or the gas is transmitted to a computing device. For example, the controller 104 and/or the computing device 108 can identify the liquid, the headspace, and/or the gas and transmit the identity of the liquid, the headspace, and/or the gas to the wine database 302. In another embodiment, the wine database 302 can provide the controller 104 and/or the computing device 108 with the identified liquid, the headspace, and/or the gas. In turn, the controller 104 and/or the computing device 108 can provide the information related to the identified liquid, the headspace, and/or the gas to the wine drinker database 304. As an alternative, the wine database 302, the controller 104, and/or the computing device 108 can provide the identity of the liquid, the headspace, and/or the gas to the wine drinker database 304. The wine drinker database 304 may then associate the identity of the liquid, the headspace, and/or the gas with a user profile so as to update the user profile.

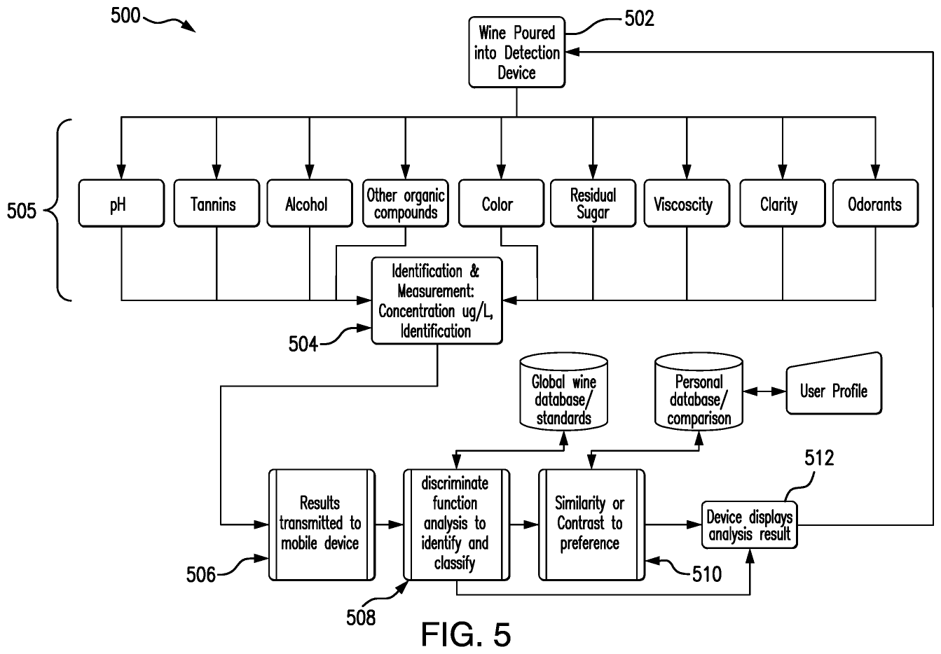

FIG. 5 is a flowchart of an example method 500. At step 502, wine is poured into a detection device (e.g., the container 102 of FIG. 1). At step 504, the device (e.g., the controller 104 and/or the sensor 106) measures a plurality of characteristics 505 of the liquid, the headspace, and/or the gas. The plurality of characteristics of the liquid, the headspace, and/or the gas can include pH, tannins, alcohol, other organic compounds, color, residual sugar, viscosity, clarity, and or odorants. At step 506, the results of the measurements can be sent to a mobile device (e.g., the computing device 108). At step 508, the mobile device can analyze the results to determine the identity of the liquid, the headspace, and/or the gas, and classify the liquid, the headspace, and/or the gas. For example, the controller 104 and/or the computing device 108 can determine from a database (e.g., the wine database 302) whether the liquid, the headspace, and/or the gas within the container matches a known wine, and/or whether the liquid, the headspace, and/or the gas within the container satisfies one or more standards associated with at least one of the known wines.

If a user associated with the mobile device has a user profile, the method continues to step 510. If the user associated with the mobile device does not have a user profile, the method continues to step 512. At step 510, the characteristics of the liquid, the headspace, and/or the gas are compared to the user's profile. For example, the characteristics of the liquid, the headspace, and/or the gas can be compared to the user's likes and dislikes to determine whether the user will like the liquid, the headspace, and/or the gas. For example, the liquid could be a Pinot Grigio, which is a white wine. The user may have a profile that indicates the user enjoys Sauvignon Blanc, which is also a white wine, but does not like Malbec, which is a bold red. Thus, the controller 104 and/or the computing device 108 can utilize the user's profile to determine that the user will probably like the Pinot Grigio because the user likes a similar wine. Alternatively, if the wine in the container was a Cabernet Sauvignon, a bold red, the controller 104 and/or the computing device 108 could determine that the user would probably dislike the Cabernet Sauvignon because the user dislikes a similar wine. Accordingly, the controller 104 and/or the computing device 108 can determine whether a user like or dislike the liquid, the headspace, and/or the gas within the container. The method continues to step 512.

At step 512, the results of the analysis are displayed. The computing device 108 can display the results to the user. For example, the computing device 108 can display the results on a display device associated with the computing device 108. As an example, the computing device 108 can be a smartphone that comprises a screen. The results can be displayed on the screen of the smartphone. The method continues back to step 502 as necessary.

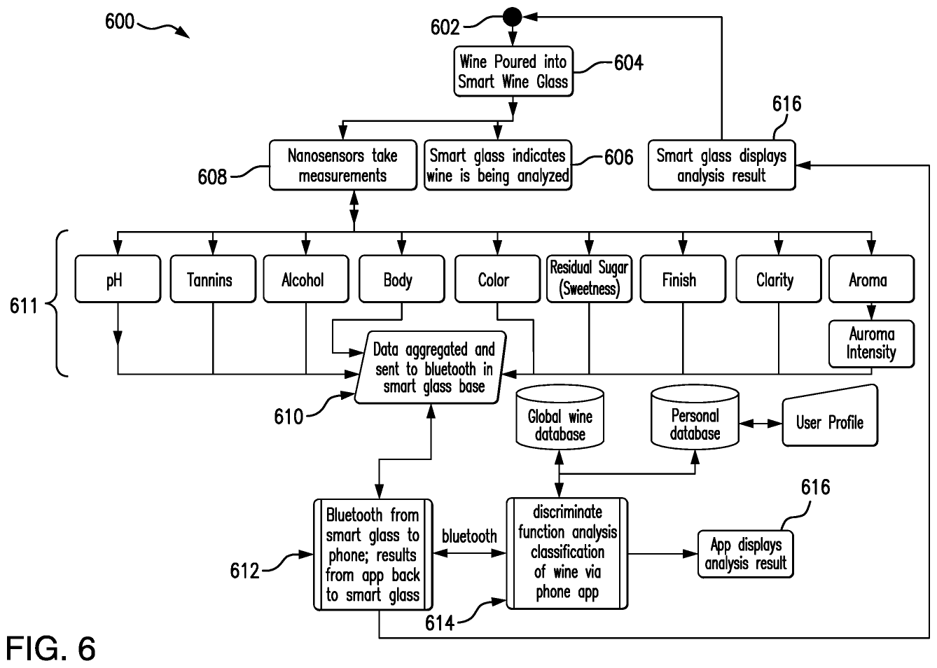

FIG. 6 is a flowchart of an example method 600. The method begins at step 602. At step 604, wine is poured into a detection device (e.g., the container 102 of FIG. 1). At step 606, the device indicates that the wine is being analyzed. For example, the device can indicate via display and/or indicators (e.g., Light Emitting Diodes (LED), a light, a sound, and so forth) that the device is analyzing the wine. At step 608, one or more sensors (e.g., the sensor 106) take one or more measurements of the wine. At step 610, the sensor aggregates the measurements and sends the data to a controller (e.g., the controller 104). The measurements can be sent via Bluetooth™, Wi-Fi, or any wireless communication.

At step 610, the device (e.g., the controller 104 and/or the sensor 106) measures a plurality of characteristics 609 of the liquid, the headspace, and/or the gas. The plurality of characteristics of the liquid, the headspace, and/or the gas can include pH, tannins, alcohol, other organic compounds, color, residual sugar, viscosity, clarity, and or odorants. The device sends the characteristics to a computing device (e.g., the computing device 108). The computing device can utilize the characteristics to determine the identity of the liquid, the headspace, and/or the gas, and classify the liquid, the headspace, and/or the gas. For example, the controller 104 and/or the computing device 108 can determine from a database (e.g., the wine database 302) whether the liquid, the headspace, and/or the gas within the container matches a known wine, and/or whether the liquid, the headspace, and/or the gas within the container satisfies one or more standards associated with at least one of the known wines.

If a user associated with the mobile device has a user profile, the characteristics of the liquid, the headspace, and/or the gas are compared to the user's profile. For example, the characteristics of the liquid, the headspace, and/or the gas can be compared to the user's likes and dislikes to determine whether the user will like the liquid within the container (e.g., the container 102). For example, the liquid could be a Pinot Grigio, which is a white wine. The user may have a profile that indicates the user enjoys Sauvignon Blanc, which is also a white wine, but does not like Malbec, which is a bold red. Thus, the controller 104 and/or the computing device 108 can utilize the user's profile to determine that the user will probably like the Pinot Grigio because the user likes a similar wine. Alternatively, if the wine in the container was a Cabernet Sauvignon, a bold red, the controller 104 and/or the computing device 108 could determine that the user would probably dislike the Cabernet Sauvignon because the user dislikes a similar wine. Accordingly, the controller 104 and/or the computing device 108 can determine whether a user like or dislike the liquid within the container. The method continues to step 614.

At step 612, the results of the analysis are displayed. The computing device 108 can display the results to the user. For example, the computing device 108 can display the results on a display device associated with the computing device 108. As an example, the computing device 108 can be a smartphone that comprises a screen. The results can be displayed on the screen of the smartphone. The method continues back to step 602 as necessary.

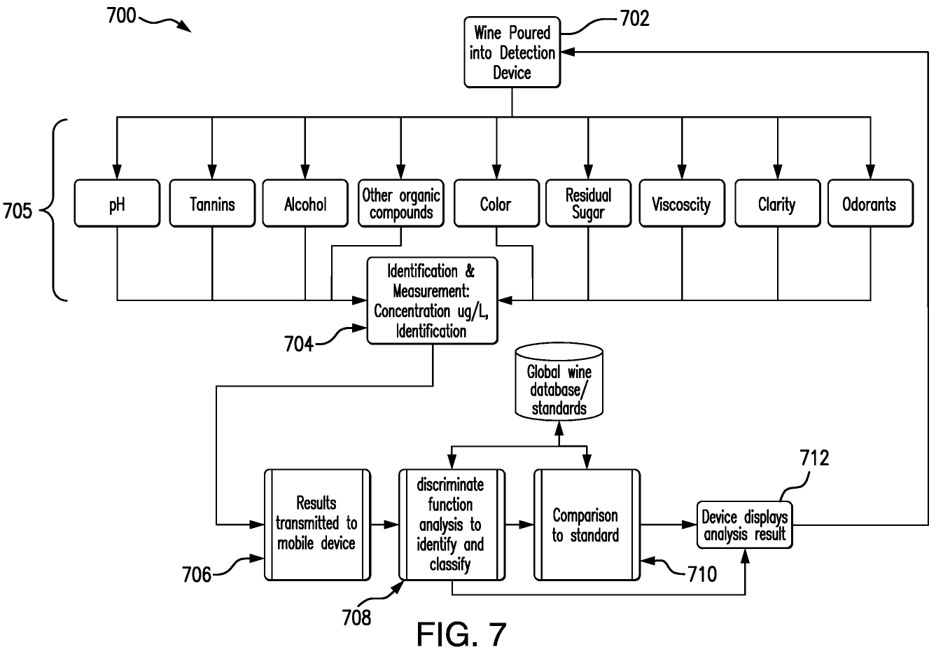

FIG. 7 is a flowchart of an example method 700. At step 702, wine is poured into a detection device (e.g., the container 102 of FIG. 1). At step 704, the device (e.g., the controller 104 and/or the sensor 106) measures a plurality of characteristics 705 of the liquid, the headspace, and/or the gas. The plurality of characteristics of the liquid, the headspace, and/or the gas can include pH, tannins, alcohol, other organic compounds, color, residual sugar, viscosity, clarity, and or odorants. At step 706, the results of the measurements can be sent to a mobile device (e.g., the computing device 108). At step 708, the mobile device can analyze the results to determine the identity of the liquid, the headspace, and/or the gas, and classify the liquid, the headspace, and/or the gas. For example, the controller 104 and/or the computing device 108 can determine from a database (e.g., the wine database 302) whether the liquid, the headspace, and/or the gas within the container matches a known wine.

If the identified wine has a standard associated with it, the method continues to step 710. If the identified wine does not have a standard associated with it, the method continues to step 712. At step 710, the identified wine can be compared to one or more standards. For example, the one or more standards can indicate the necessary characteristics for a wine to have a certain label (e.g., Champagne). The characteristics of the wine can be compared to the one or more standards to determine whether the meets the one or more standards. For example, the computing device can determine whether the characteristics of the wine meet the one or more standards, and determine results based on the comparison.

At step 712, the results of the analysis are displayed. The computing device 108 can display the results to the user. For example, the computing device 108 can display the results on a display device associated with the computing device 108. As an example, the computing device 108 can be a smartphone that comprises a screen. The results can be displayed on the screen of the smartphone. The method continues back to step 702 as necessary.

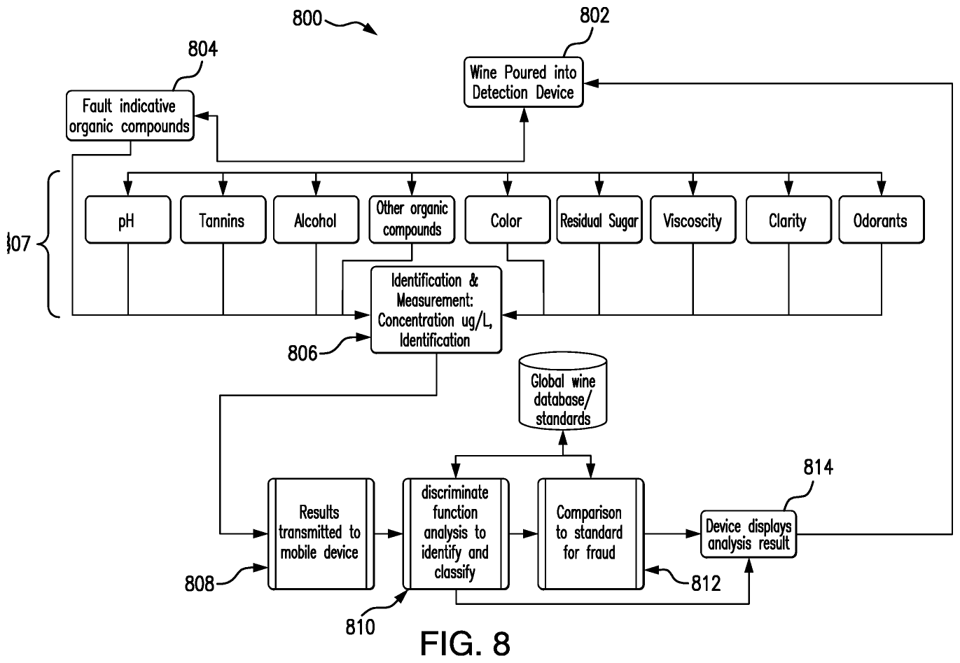

FIG. 8 is a flowchart of an example method 800. At step 802, wine is poured into a detection device (e.g., the container 102 of FIG. 1). At step 804, an organic compound that indicates a fault can be detected. At step 806, the device (e.g., the controller 104 and/or the sensor 106) measures a plurality of characteristics 807 of the liquid, the headspace, and/or the gas. The plurality of characteristics of the liquid, the headspace, and/or the gas can include pH, tannins, alcohol, other organic compounds, color, residual sugar, viscosity, clarity, and or odorants. At step 808, the results of the measurements can be sent to a mobile device (e.g., the computing device 108). At step 810, the mobile device can analyze the results to determine the identity of the liquid, the headspace, and/or the gas, and classify the liquid, the headspace, and/or the gas. For example, the controller 104 and/or the computing device 108 can determine from a database (e.g., the wine database 302) whether the liquid, the headspace, and/or the gas within the container matches a known wine.

If the identified wine has a standard associated with it, the method continues to step 812. If the identified wine does not have a standard associated with it, the method continues to step 814. At step 812, the identified wine can be compared to one or more standards for fraud. For example, the one or more standards can indicate the necessary characteristics for a wine to have a certain label (e.g., Champagne). The characteristics of the wine can be compared to the one or more standards to determine whether the meets the one or more standards. For example, the computing device can determine whether the characteristics of the wine meet the one or more standards, and determine results based on the comparison. If the liquid, the headspace, and/or the gas does not satisfy one or more of the standards, the computing device can determine that the wine does not meet the standard and indicate that the wine is a fraud.

At step 814, the results of the analysis are displayed. The computing device 108 can display the results to the user. For example, the computing device 108 can display the results on a display device associated with the computing device 108. As an example, the computing device 108 can be a smartphone that comprises a screen. The results can be displayed on the screen of the smartphone. The method continues back to step 802 as necessary.

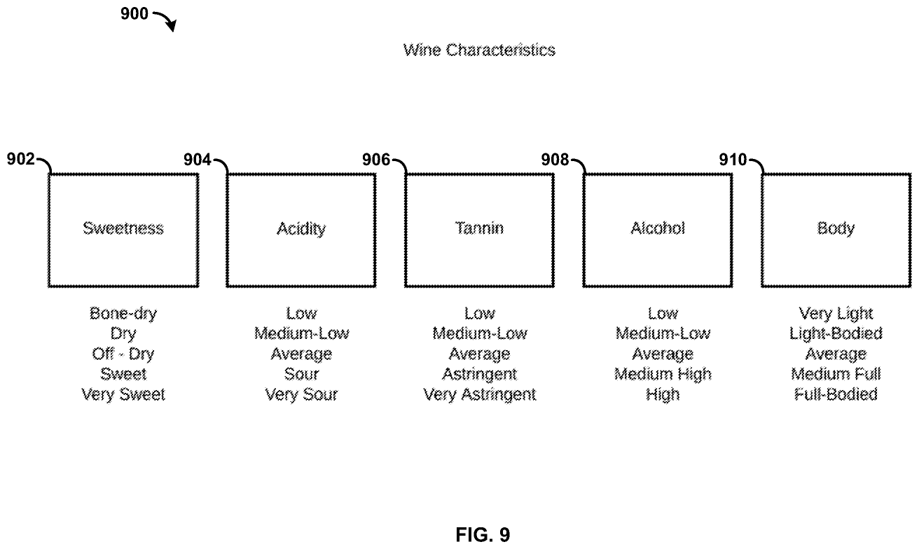

FIG. 9 is a diagram illustrating wine characteristics 900. Specifically, the wine characteristics 900 can include sweetness 902, acidity 904, tannin 906, alcohol 908, and body 910. The sweetness 902 can indicate that the wine is bone-dry, dry, off-dry, sweet, and/or very sweet. The sweetness 902 can be determine based on measuring the sugar content of the wine. The acidity 904 can indicate that the wine has an acidity level that is low, medium-low, average, sour, and/or very sour. The acidity 904 can be determined based on the pH of the wine. The tannin 906 can indicate that the wine has a tannin level that is low, medium-low, average, astringent, and/or very astringent. The alcohol 908 can indicate the alcohol of the wine. The alcohol 908 can be low, medium-low, average, medium-high, and/or high. The alcohol 908 can be determined by measuring the amount of alcohol by volume (ABV) in the wine. The body 910 can indicate the body of the wine. The body 910 can be very light, light-bodied, average, medium full, and/or full-bodied.

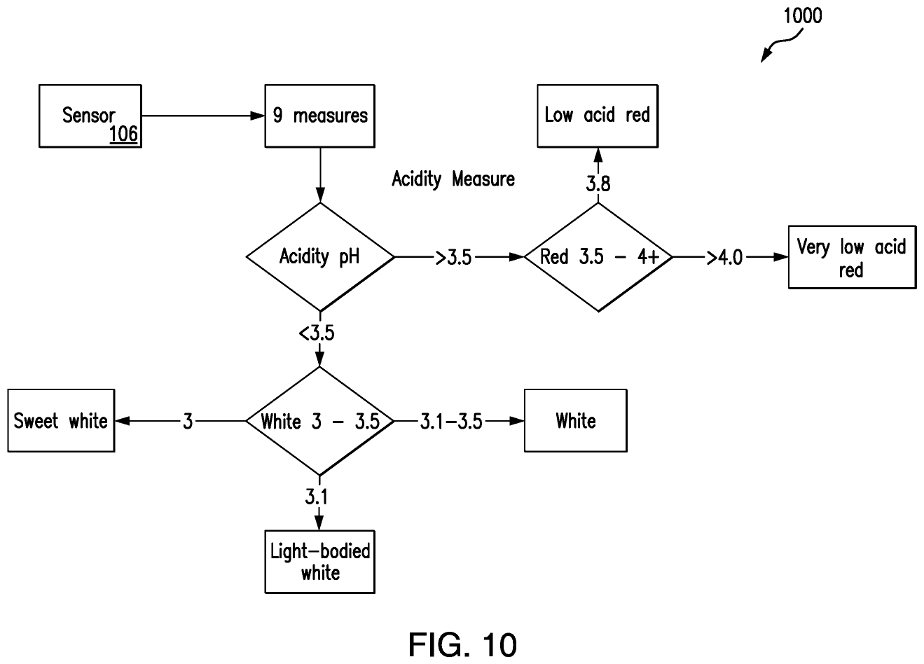

FIG. 10 is a diagram illustrating wine acidity 1000. Specifically, the wine acidity 1000 illustrates a determination that a computing device (e.g., the controller 104 and/or the computing device 108) can make to identify a wine based on the acidity. For example, a sensor (e.g., the sensor 106) can take 9 measurements to measure the pH of the wine. If the wine is above a 3.5 pH, the wine is a red wine. If the pH is between a 3.8 and 4.0 pH, the wine is a low acid red wine. If the pH is greater than 4.0 pH, the wine is a very low acid red. If the wine is below a 3.5 pH, the wine is a white wine. If the wine is a pH of 3, the wine is a sweet white wine. If the pH is 3.1, the wine is a light-bodied white wine. If the pH is between 3.1-3.5, the wine is a non-sweet white wine. Thus, the computing device can determine the wine based on the acidity of the wine. The computing device can utilize the acidity of the wine 1000 to facilitate identifying the wine.

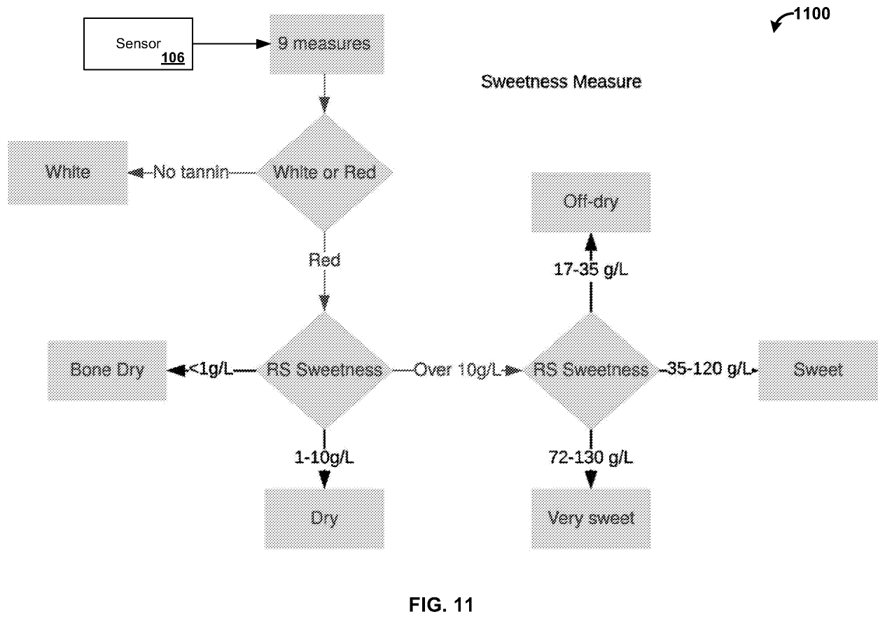

FIG. 11 is a diagram illustrating wine sweetness 1100. Specifically, the wine acidity 1100 illustrates a determination that a computing device (e.g., the controller 104 and/or the computing device 108) can make to identify a wine based on the sweetness of the wine. For example, a sensor (e.g., the sensor 106) can take 9 measurements to measure the sugar content of the wine. First, a determination is made if the wine is a white or a red wine. If the wine is a white wine, the sweetness measure is not taken because the white wine does not have tannins. Rather, as explained above, the sweetness of the white wine is determined based on the acidity of the wine. If the wine is a red wine, the sweetness is measured in.

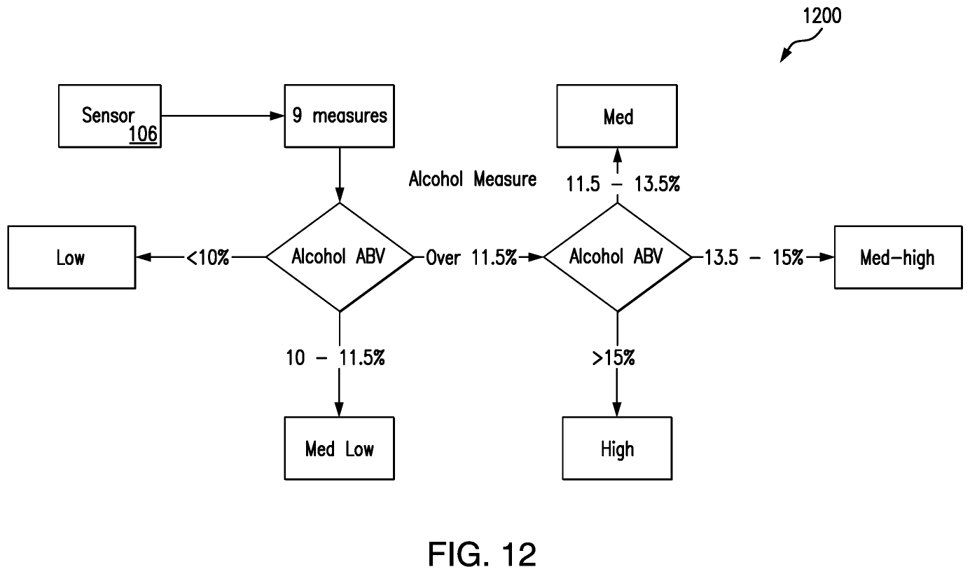

FIG. 12 is a diagram illustrating wine alcohol measurements 1200. Specifically, the wine acidity 1200 illustrates a determination that a computing device (e.g., the controller 104 and/or the computing device 108) can make to identify a wine based on the alcohol content of the wine. For example, a sensor (e.g., the sensor 106) can take 9 measurements to measure the ABV of the wine. If the ABV is below 10%, the wine has a low alcohol content. If the ABV is between 10% and 11.5%, the wine has a medium low alcohol content. If the ABV is between 11.5% and 13.5%, the wine has a medium alcohol content. If the ABV is between 13.5% and 15%, the wine has a medium-high alcohol content. If the ABV is greater than 15%, the wine has a high alcohol content. The computing device can utilize the alcohol of the wine 1200 to facilitate identifying the wine.

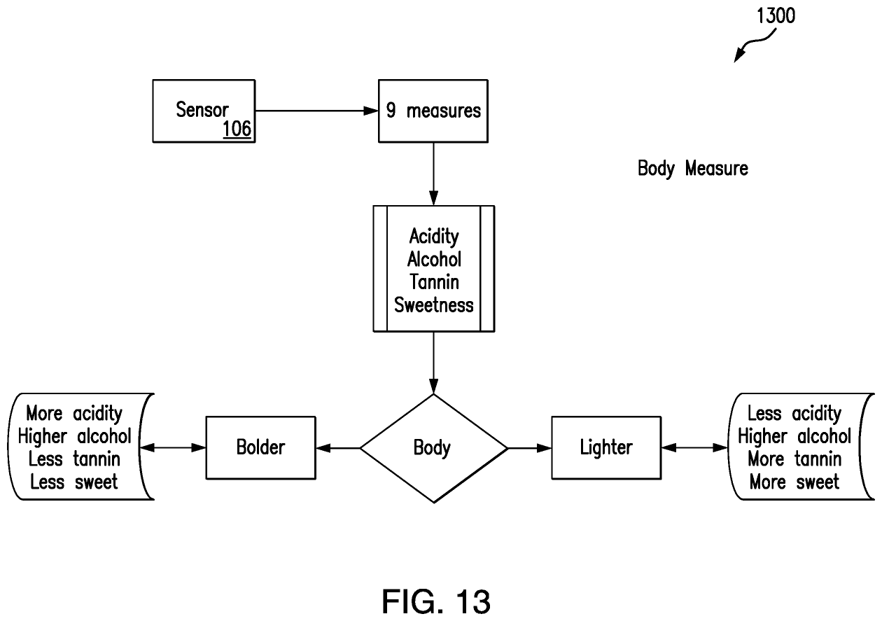

FIG. 13 is a diagram illustrating body characteristics of a wine 1300. For example, the computing device can determine the body characteristics of the wine 1300 after determining the acidity (e.g., based on the wine acidity 1000 of FIG. 10), the alcohol (e.g., based on the alcohol measurements 1200 of FIG. 12), the tannin, and the sweetness (e.g., based on the sweetness 1100 of FIG. 11) of the wine. Based on the acidity, the alcohol, the tannin, and the sweetness of the wine, the computing device can determine the body. As an example, the higher the acidity, the higher the alcohol, the less tannin, and the less sweet the wine is, the bolder the wine will be. Conversely, the lower the acidity, the higher the alcohol, the higher the tannin, and the higher the sweetness of the wine, the less bold the wine will be. The computing device can determine the body based on a scale. For example, the scale could be from 1 to 10, with 1 being the boldest a wine could be and with 10 being the least bold a wine could be. The computing device can utilize the body characteristics of the wine 1300 to facilitate identifying the wine.

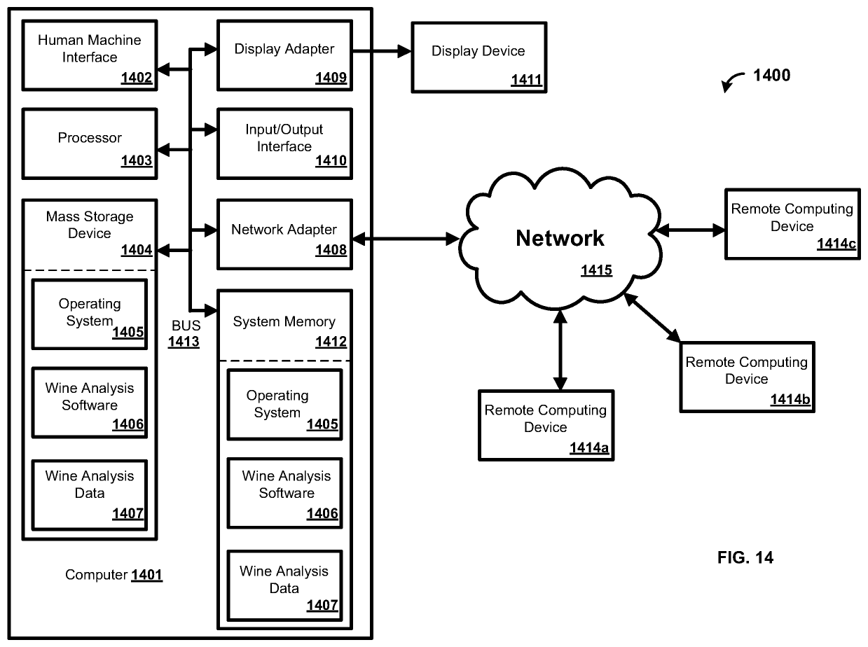

FIG. 14 shows an example of an operating environment 1400 a computer 1401. While the computing device 108 is shown for ease of explanation, it is to be understood that the controller 102, the wine database 302, the wine drinker database 304, and/or the sensor 106 can include any and all of the functionality described below. The operating environment 1400 is only an example of an operating environment and is not intended to suggest any limitation as to the scope of use or functionality of operating environment architecture. Neither should the operating environment 1400 be interpreted as having any dependency or requirement relating to any one or combination of components illustrated in the operating environment 1400.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise programmable consumer electronics, network PCs, minicomputers, mainframe computers, smart devices, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computing device 1401. The computing device 1401 can comprise one or more components, such as one or more processors 1403, a system memory 1412, and a bus 1413 that couples various components of the computing device 1401 including the one or more processors 1403 to the system memory 1412. In the case of multiple processors 1403, the system can utilize parallel computing.

The bus 1413 can comprise one or more of several possible types of bus structures, such as a memory bus, memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 1413, and all buses specified in this description can also be implemented over a wired or wireless network connection and one or more of the components of the computing device 1401, such as the one or more processors 1403, a mass storage device 1404, an operating system 1405, wine analysis software 1406, wine analysis data 1407, a network adapter 1408, a system memory 1412, an Input/Output Interface 1410, a display adapter 1409, a display device 1411, and a human machine interface 1402, can be contained within one or more remote computing devices 1414a,b,c at physically separate locations, connected through buses of this form, in effect implementing a fully distributed system.

The computing device 1401 typically comprises a variety of computer readable media. As an example, readable media can be any available media that is accessible by the computing device 1401 and comprises, for example and not meant to be limiting, both volatile and non-volatile media, removable and non-removable media. The system memory 1412 can comprise computer readable media in the form of volatile memory, such as random access memory (RAM), and/or non-volatile memory, such as read only memory (ROM). The system memory 1412 typically can comprise data such as wine analysis data 1407 and/or program modules such as operating system 1405 and wine analysis software 1406 that are accessible to and/or are operated on by the one or more processors 1403.

In another example, the computing device 1401 can also comprise other removable/non-removable, volatile/non-volatile computer storage media. The mass storage device 1404 can provide non-volatile storage of computer code, computer readable instructions, data structures, program modules, and other data for the computing device 1401. For example, a mass storage device 1404 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1404, including by way of example, an operating system 1405 and wine analysis software 1406. One or more of the operating system 1405 and the wine analysis software 1406 (or some combination thereof) can comprise program modules. The wine analysis data 1407 can also be stored on the mass storage device 1404. The wine analysis data 1407 can be stored in any of one or more databases known in the art. Examples of such databases comprise, DB2®, Microsoft® Access, Microsoft® SQL Server, Oracle®, MySQL, PostgreSQL, and the like. The databases can be centralized or distributed across multiple locations within the network 1415.

In one example, the wine analysis software 1406 includes the functionality to operate the controller 104 and/or the sensor 106. For example, the wine analysis software 1406 includes the functionality to communicate with the controller 104 and provide operational instructions and/or preferences to the controller 104. As an example, the wine analysis software 1406 can receive data from the sensor 106, and the wine analysis software 1406 can use the data to determine how the sensor 106 should be controlled. The wine analysis software 1406 can instruct the controller 104 to selectively activate the sensor 106. The wine analysis software 1406 can instruct the controller 104 to automatically activate the sensor 106. For example, the wine analysis software 1406 can instruct the controller 104 to activate the sensor 106 to determine characteristics of a liquid, a headspace, and/or a gas upon the liquid, the headspace, and/or the gas being detected within the container 102. As another example, the wine analysis software 1406 can receive input from a user that instructs the wine analysis software 1406 to have the controller 104 activate measurement of the liquid, the headspace, and/or the gas using the sensor 106.

As another example, the wine analysis software 1406 can provide settings to the controller 104 that indicate when the controller 104 should do an analysis of the liquid, the headspace, and/or the gas in the container 104. As one example, the wine analysis software 1406 can provide start and stop times that the controller 104 should activate the sensor 106. As another example, the wine analysis software 1406 can indicate times that the controller 104 should start dynamically managing the sensor 106. As a further example, the wine analysis software 1406 can provide settings as to when the controller 104 should perform a measurement using the sensor 106. In one example, a user of the wine analysis software 1406 actively selects the instructions or settings that are transmitted to the controller 104. In another example, the wine analysis software 1406 dynamically decides the instructions or settings that are transmitted to the controller 104 without input from a user. In another example, the wine analysis software 1406 receives input from a user indicating the preferences and/or settings the user would like the wine analysis software 1406 to implement. The wine analysis software 1406 can then automatically transmit instructions to the controller 104 based on the user indicated preferences and/or settings. In one example, the user of the wine analysis software 1406 selects specific settings related to a measurement using the sensor 106.

In one example, the wine analysis software 1406 can run data analysis on the measurements of the sensor 106. For example, the sensor 106 can provide instantaneous output signals. The wine analysis software 1406 can store the output signals from the sensor 106 and convert the output signals into a data.

In one example, the wine analysis software 1406 is a web based, telecommunications based, or smart device application that has an associated interface that a user can access which controls the functionality of the controller 104 and the sensor 106.

In another example, the user can enter commands and information into the computing device 1401 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices can be connected to the one or more processors 1403 via a human machine interface 1402 that is coupled to the bus 1413, but can be connected by other interface and bus structures, such as a parallel port, game port, an IEEE 1394 Port (also known as a Firewire port), a serial port, network adapter 1408, and/or a universal serial bus (USB).

In yet another example, a display device 1411 can also be connected to the bus 1413 via an interface, such as a display adapter 1409. It is contemplated that the computing device 1401 can have more than one display adapter 1409 and the computing device 1401 can have more than one display device 1411. For example, a display device 1411 can be a monitor, an LCD (Liquid Crystal Display), light emitting diode (LED) display, television, smart lens, smart glass, smart container, display of a smart device, and/or a projector. In addition to the display device 1411, other output peripheral devices can comprise components such as speakers (not shown) and a printer (not shown) which can be connected to the computing device 1401 via Input/Output Interface 1410. Any step and/or result of the methods can be output in any form to an output device. Such output can be any form of visual representation, including, but not limited to, textual, graphical, animation, audio, tactile, and the like. The display 1411 and the computing device 1401 can be part of one device, or separate devices.

The computing device 1401 can operate in a networked environment using logical connections to one or more remote computing devices 1414a,b,c. By way of example, a remote computing device 1414a,b,c can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. As an example, remote computing devices 1414a,b,c can be the controller 104, the computing device 108, and the sensor 106. Logical connections between the computing device 1401 and a remote computing device 1414a,b,c can be made via a network 1415, such as a local area network (LAN) and/or a general wide area network (WAN). Such network connections can be through a network adapter 1408. A network adapter 1408 can be implemented in both wired and wireless environments. Such networking environments are conventional and commonplace in dwellings, offices, enterprise-wide computer networks, intranets, and the Internet. The network 1415 can also comprise a Bluetooth™ or Wi-Fi.

For purposes of illustration, application programs and other executable program components such as the operating system 1405 are shown herein as discrete blocks, although it is recognized that such programs and components can reside at various times in different storage components of the computing device 1401, and are executed by the one or more processors 1403 of the computing device 1401. An implementation of wine analysis software 1406 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with specific examples, it is not intended that the scope be limited to the particular examples set forth, as the examples herein are intended in all respects to be possible examples rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of examples described in the specification.

It will be apparent to those skilled in the art that various modifications and variations can be made without departing from the scope or spirit. Other examples will be apparent to those skilled in the art from consideration of the specification and practice disclosed herein. It is intended that the specification and examples be considered as exemplary only, with a true scope and spirit being indicated by the following claims.

1. A system, comprising:
a container comprising
one or more sensors configured to take a plurality of measurements of the liquid within the container; and
a controller, in communication with the one or more sensors, wherein the controller is configured to,
receive, from the one or more sensors, data indicative of the plurality of measurements of the liquid,
determine, based on the data, a plurality of characteristics of the liquid,
transmit, to a computing device, the plurality of characteristics of the liquid.

2. The system of claim 1, wherein the liquid comprises one or more of: the liquid, a headspace above the liquid, or a gas associated with the liquid.

3. The system of claim 1, wherein the computing device is configured to store the plurality of characteristics of the liquid in a database.

4. The system of claim 1, wherein the computing device is configured to match the plurality of characteristics of the liquid with a secondary database of characteristics of known liquids.

5. The system of claim 1, wherein the computing device is configured to add the identity of the liquid to a profile associated with a drinker of the liquid.

6. The system of claim 5, wherein the computing device is further configured to determine, based on a machine learning algorithm and the profile of the drinker, one or more types of liquid.

7. The system of claim 1, wherein the one or more sensors are miniaturized sensors that are configured to determine at least one of: organic or inorganic chemicals within the liquid, a pH of the liquid, tannins of the liquid, an alcohol content of the liquid, a body of the liquid, a color of the liquid, a sweetness of the liquid, a finish of the liquid, a clarity of the liquid, or an aroma of the liquid.

8. The system of claim 1, wherein the computing device comprises a smartphone.

9. An apparatus, comprising:
one or more processors; and
a memory storing processor executable instructions that, when executed by the one or more processors, cause the apparatus to:
receive, from one or more sensors, data indicative of a plurality of measurements of a liquid within a container,
determine, based on the received data, a plurality of characteristics of the liquid,
determine, based on the plurality of characteristics of the liquid, an identity of the liquid, wherein the identity comprises: a type of the liquid, a manufacturer of the liquid, and a year the liquid was manufactured, and
transmit, to a computing device, the identity of the liquid.

10. The apparatus of claim 9, wherein the liquid comprises one or more of: the liquid, a headspace above the liquid, or a gas associated with the liquid.

11. The apparatus of claim 9, wherein the computing device is configured to store the identity of the liquid in a database.

12. The apparatus of claim 9, wherein the computing device is configured to add the identity of the liquid to a profile associated with a drinker of the liquid.

13. The apparatus of claim 12, wherein the computing device is further configured to determine, based on a machine learning algorithm and the profile of the drinker, one or more types of liquid.

14. The apparatus of claim 9, wherein the one or more sensors are miniaturized sensors that are configured to determine and measure at least one of: organic or inorganic chemicals within the liquid, the presence or absence of undesirable chemicals within the liquid, a least one of pH of the liquid, tannins of the liquid, an alcohol content of the liquid, a body of the liquid, a color of the liquid, a sweetness of the liquid, a finish of the liquid, a clarity of the liquid, or an aroma of the liquid.

15. The apparatus of claim 9, wherein the computing device comprises a smartphone.

16. A method comprising:
receiving, from one or more sensors, data indicative of a plurality of measurements of a liquid within a container,
determining, based on the data, a plurality of characteristics of the liquid,
determining, based on the plurality of characteristics of the liquid, an identity of the liquid, wherein the identity comprises: a type of the liquid, a manufacturer of the liquid, and a year the liquid was manufactured, and
transmitting, to a computing device, the identity of the liquid.

17. The method of claim 16, wherein the liquid comprises one or more of: the liquid, a headspace above the liquid, or a gas associated with the liquid.

18. The method of claim 16, wherein the liquid comprises a wine, wherein the computing device is further configured to store the identity of the wine in a database, and wherein the computing device is further configured to add the identity of the wine to a profile associated with a drinker of the wine.

19. The method of claim 18, wherein the computing device is further configured to determine, based on a machine learning algorithm and the profile of the drinker, one or more types of liquid.

20. The method of claim 16, wherein the one or more sensors are miniaturized sensors that are configured to determine at least one of: organic or inorganic chemicals within the liquid, a pH of the liquid, tannins of the liquid, an alcohol content of the liquid, a body of the liquid, a color of the liquid, a sweetness of the liquid, a finish of the liquid, a clarity of the liquid, or an aroma of the liquid.
